(19) United States
(12) Patent Application Publication
(10) Pub. No.: US 2013/0122579 A1
(43) Pub. Date: May 16, 2013

(54) FERMENTER FOR A BIOGAS PLANT

(71) Applicant: Eric Priller, Thierstein/Schwarzhammer (DE)

(72) Inventor: Eric Priller, Thierstein/Schwarzhammer (DE)

(73) Assignee: OAG OBJEKT- UND ANLAGENPLANUNGS - GESEKKSCHAFT MBH, Plauen (DE)

(21) Appl. No.: 13/255,534

(22) PCT Filed: Mar. 3, 2010
(86) PCT No.: PCT/EP2010/001321

§ 371 Date: Sep. 25, 2011

(30) Foreign Application Priority Data
Mar. 10, 2009 (DE) 10 2009 012 418.7

(51) Int. Cl. C12M 1/00

(52) U.S. Cl. CPC C12M 21/00
USPC 435/289.1

(57) ABSTRACT

A fermenter for a biogas plant which operates with wet fermentation includes a fixed casing which surrounds a reaction space, an inlet for biomass and an outlet for the fermentation reside. A wall is arranged inside the casing, which separates the inlet from the outlet.

### RELATED APPLICATION

This is the U.S. national phase filing of PCT/EP2010/001321, filed Mar. 3, 2010, which claims priority to German Application Serial No. 10 2009 012 418.7, filed Mar. 10, 2009.

### TECHNICAL FIELD

This invention relates to a fermenter for a biogas plant which operates with wet fermentation (up to 15% dry matter), comprising a fixed casing which surrounds a reaction space, an inlet for biomass and an outlet for the fermentation residue.

### BACKGROUND OF THE INVENTION

In the fermenter, the biomass is fermented under anaerobic conditions, wherein biogas is obtained. With regard to an optimum efficiency it is desirable when the biomass introduced into the fermenter only leaves the same when it is fermented completely. In practice, however, this can only be achieved with great difficulty. In the usually employed fermenters, which operate by the wet fermentation process, the problem exists that during removal of the fermentation residue, some unfermented biomass always also leaves the fermenter (“loss of fresh weight”). This loss of fresh weight could not be avoided in the previously used fermenters, which operate by a pot process. Fermenters should be improved such that the loss of fresh weight is reduced or even avoided completely.

### SUMMARY OF THE INVENTION

A fermenter is formed inside a casing a wall to separate the inlet from the outlet. This prevents freshly introduced biomass from directly reaching the outlet such that the biomass cannot leave the fermenter without being fermented completely.

In one example, the casing wall extends spirally. In this way, a comparatively long path can be defined inside the fermenter, which the biomass must cover from the inlet to the outlet.

In accordance with an exemplary embodiment of the invention it is provided that the wall defines a spiral path for the biomass inside the reaction space, wherein the inlet is arranged at one end of the path and the outlet is arranged at the other end. When the biomass is supplied continuously, this design allows adjusting a slow movement of the biomass along the path towards the outlet, with the retention time of the biomass in the fermenter being determined such that the biomass is fermented completely when it reaches the outlet. In this way, an optimum efficiency is obtained, as on the one hand no unfermented biomass can leave the fermenter and on the other hand biomass fermented already does not stay in the fermenter for an excessively long period, occupying volume therein which might otherwise be utilized for the fermentation process.

### BRIEF DESCRIPTION OF THE DRAWINGS

The invention will be described below with reference to a preferred embodiment which is represented in the attached drawings, in which:

FIG. 1 shows a cross-section through a fermenter according to the invention; and

FIG. 2 shows a section along plane II-II of FIG. 1.

### DETAILED DESCRIPTION

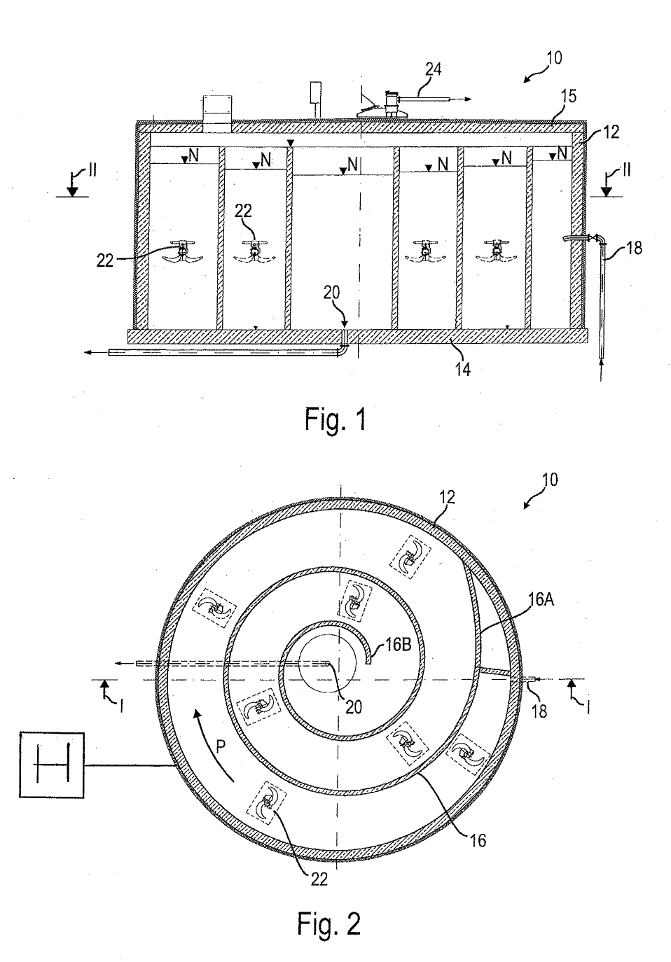

FIGS. 1 and 2 show a fermenter 10 which is part of a biogas plant. The fermenter 10 is formed by a fixed casing 12 which is arranged on a foundation 14. The casing 12 is cylindrical, with the central axis of the cylinder being vertical to the foundation 14. In the interior of the casing 12 a wall 16 is arranged, which extends spirally. An outer end 16A of the wall 16 touches the casing 12, whereas the inner end 16B of the wall 16 ends in the vicinity of the central axis of the cylindrical casing 12. By the spiral wall 16 a path is formed which extends helically from the outside, i.e. from the casing 12, towards the center. This path extends in a vertical direction from the upper edge of the wall 16 down to the foundation 14. On the upper surface of the casing 12 a lid 15 is arranged, so that in the interior of the casing 12 a sealed reaction space is formed. The lid 15 is located at a distance from the upper edge of the wall 16, so that in the upper region of the fermenter a continuous gas collecting space is formed.

Both the casing 12 and the spiral wall 16 can be made of metal and/or concrete. The lid 15 here also is a heavy construction. Alternatively, a plastic hood can also be used.

In the vicinity of the outer end 16A of the spiral wall 16 an inlet 18 for biomass is arranged, which extends through the casing 12. Approximately in the middle of the reaction space of the fermenter enclosed by the casing 12 and hence in the interior of the spiral formed by the wall 16, an outlet 20 is arranged for the fermentation residue, i.e. for the fermented biomass. Due to the spiral wall 16, the biomass moves along a predetermined path through the fermenter, in order to get from the inlet 18 to the outlet 20. Along the path a plurality of stirrers 22 are arranged, which ensure a uniform vertical intermixing. Furthermore, the fermenter is provided with a heater H, which ensures a constant temperature in the interior of the fermenter.

In operation, biomass is supplied to the fermenter via the inlet 18. Corresponding to the quantity supplied, completely fermented biomass is discharged through the outlet as fermentation residue. Therefore, a very slow flow of the biomass is obtained from the inlet 18 to the outlet 20 in direction of the arrow P of FIG. 2. The resulting different levels of the biomass along the path formed are indicated in FIG. 1 with the arrows N. While proceeding from the inlet 18 to the outlet 20, the biomass is regularly circulated by the stirrers 22 in a vertical direction, so that the fermentation process proceeds uniformly. The biogas obtained during the fermentation is removed via a gas vent 24.

Although an embodiment of this invention has been disclosed, a worker of ordinary skill in this art would recognize that certain modifications would come within the scope of this invention. For that reason, the following claims should be studied to determine the true scope and content of this invention.

1. A fermenter for a biogas plant which operates with wet fermentation, comprising:
a fixed casing which surrounds a reaction space;
an inlet for biomass; and
an outlet for fermentation residue, wherein a wall is arranged inside the fixed casing, which separates the inlet from the outlet.

2. The fermenter according to claim 1, wherein the wall extends spirally.

3. The fermenter according to claim 2, wherein the wall defines a spiral path for the biomass inside the reaction space, and wherein the inlet is arranged at one end of the spiral path and the outlet is arranged at the other end of the spiral path.

4. The fermenter according to claim 3, wherein the inlet is arranged at an outer end of the path.

5. The fermenter according to claim 1, wherein the outlet is arranged approximately centrally at a bottom of the fermenter.

6. The fermenter according to claim 1, wherein the fixed casing and the wall extend vertically.

7. The fermenter according to claim 1, wherein a plurality of stirrers are arranged along a path defined by the wall.

8. The fermenter according to claim 1, wherein a heater is provided.

9. The fermenter according to claim 1, wherein the fixed casing is cylindrical.
